SEQUENCE LISTING (5 sequences)

SEQ ID NO: 1 - DNA, 17 bp, Artificial
  Forward Primer
cctacgggaggcagcag

SEQ ID NO: 2 - DNA, 17 bp, Artificial
  Reverse Primer
attaccgcggctgctgg

SEQ ID NO: 3 - DNA, 20 bp, Artificial
  Forward Primer
agagtttgatcctggctcag

SEQ ID NO: 4 - DNA, 18 bp, Artificial
  Reverse Primer
aaaggaggtgatccagcc

SEQ ID NO: 5 - DNA, 19 bp, Artificial
  Primer
gtattaccgcggctgctgg

(19) United States
(12) Patent Application Publication
(10) Pub. No.: US 2008/0166770 A1
(43) Pub. Date: Jul. 10, 2008

(54) METHOD AND APPARATUS FOR AMPLIFYING AND SYNTHESISIZING NUCLEIC ACID WITH DENATURANT

(75) Inventors: Tomoyuki MORITA, Fujisawa-shi (JP); Takashi Matsumura, Palo Alto, CA (US); Akiko Miya, Kamakura-shi (JP); Motohiko Nohmi, Yokohama-shi (JP); Shunsuke Shimizu, Yokohama-shi (JP)

Correspondence Address: BROWDY AND NEIMARK, P.L.L.C.;624 NINTH STREET, NW, SUITE 300, WASHINGTON, DC, 20001-5303, US

(73) Assignee: EBARA Corp., Tokyo (JP)

(21) Appl. No.: 11/864,142

(22) Filed: Sep. 28, 2007

Related U.S. Application Data
(60) Provisional application No. 60/847,915, filed on Sep. 29, 2006.

(51) Int. Cl. C12P 19/34

(52) U.S. Cl. 435/91.1

(57) ABSTRACT

The invention relates generally to the field of treating a nucleic acid. More particularly, the invention provides a method for amplifying a nucleic acid and an apparatus for amplifying a nucleic acid, as well as method and apparatus for synthesizing nucleic acid to be used for the amplification.

### BACKGROUND OF THE INVENTION

The invention relates generally to the field of treating a nucleic acid. More particularly, the invention provides a method for amplifying a nucleic acid and an apparatus for amplifying a nucleic acid, as well as method and apparatus for synthesizing nucleic acid to be used for the amplification.

Polymerase chain reaction (PCR) is one of the most important techniques in biological analysis. PCR played a crucial role in the Human Genome Project and is still indispensable in post-genome era where individual genomic information needs to be obtained. However, such analyses of nucleic acids are still relatively expensive and can not be obtained fast enough to apply to individuals. PCR is one of the most expensive and time-consuming procedures, because PCR requires expensive reagents (especially thermostable DNA polymerase) and time-consuming thermal cycling. There is a great demand for a cheaper and faster alternative.

PCR was first developed in 1985 by Mullis [Science, 20 Dec. 1985, v. 230 (4732): 1350-1354]. PCR, typically requires thermal cycling of three steps; denaturing, annealing and extension. These steps are typically repeated between 25 and 40 times. The reaction buffer contains a thermostable DNA polymerase, a pair of oligonucleotide primers, deoxynucleotide triphosphates (dNTPs), MgCl₂ and KCl. MgCl₂ is essential for DNA polymerase activity and KCl helps to control appropriate denaturing DNA polymerases used for PCR should be thermostable to maintain its activity through thermal cycling. This fact also increases the cost of PCR because thermostable DNA polymerases are relatively expensive.

Lab-on-a-chip (LOC) technology, which integrate (multiple) laboratory functions on a single chip of only millimeters to a few square centimeters in size, can provide solutions for cheaper and faster PCR because of its potential to reduce analysis time and consumption of reagents. On-chip PCR has been the subject of extensive research over the past decade [Anal Bioanal Chem (2003) 377: 820-825, Lab Chip, 2004, 4, 534-546, Anal. Chem. 2005, 77, 3867-3694]. On-chip PCR is also called PCR chip. There are two types of PCR chips; simple-well and continuous-flow type. The simple-well type of PCR chip consists of small-volume reaction vessels in which PCR is performed. The continuous-flow type of PCR chip consists of a serpentine channel and three thermal-controlled regions. The mixture of a sample and reaction buffer can repeatedly pass three temperature-controlled regions through the channel, and as a result, PCR reaction occurs. The performance of PCR chip is superior to traditional PCR in terms of speed, throughput and reaction volume.

The simple-well type of PCR chip is just a miniaturization of reaction volume. A typical example is the making of microchambers on silicon water with micromachining technique [Anal. Chem. 2004, 76, 6434-6439]. With this type of PCR chip it is easy to decrease a reaction volume, array a number of reaction wells, and increase throughput as a result. Moreover, it is easy to integrate PCR and CE on a microchip [Anal. Chem. 1996, 68, 4081-4086, Anal. Chem. 1998, 70, 158-162, Anal. Chem. 2004, 76, 3162-3170]. However, thermal cycling of an entire microchip requires the time scale of PCR reaction comparable to traditional PCR methods. Therefore, a miniaturized heater and a temperature sensor were fabricated on a microchip, thereby achieving a fast temperature transition between each step [Anal. Chem. 2004, 76, 3162-3170]. However, these chips need a relatively complex fabrication process that in turn increases production costs. PCR chips to be used are disposable so as to prevent cross contamination.

The continuous-flow type PCR chip was first reported by Kopp et al. in 1998 [SCIENCE, VOL. 280, 15 MAY, 1998, 1046-1048]. This type of PCR chip consists of a serpentine channel and three different temperature regions. The mixture of a sample and reagents repeatedly passes the three regions through the serpentine channel. The three regions are controlled to the temperatures that are required for denaturing, annealing and extension. This type of PCR chip does not require thermal transition of the entire chip. Therefore, the reaction is very fast, as a result minimizing non-specific amplification. However, the number of cycles is fixed. The number of cycles is pre-determined by microchannel design and cannot be changed. Parallelization is also difficult.

### PRIOR ART

Still, there is a room to be improved for the simple-well and continuous-flow type of PCR chip as mentioned above, resulting in modifications of PCR chips so far. For example, Liu et al. reported a rotary device for PCR [Electrophoresis 2002, 23, 1531-1536]; the mixture of a sample and reagents can rotate between three different temperature regions using this device. Krishnan et al. demonstrated PCR in a Rayleigh-Benard convection cell [SCIENCE VOL 298 25 OCT. 2002]. Chen et al. reported electrokinetically synchronized PCR chip [Anal. Chem. 2005, 77, 658-666], where each of four electrodes is placed at four different each corners of a single loop. Once a sample is injected into the loop, an electric field is applied between two opposite corners of the loop. The section of the loop in which the field is applied is synchronized with the sample position to allow cycling the sample through the single-loop channel.

On the other hand, Knapp et al. and Ogiwara et al. proposed PCR without thermal cycling. Knapp et al. (Japanese Domestic Patent Publication NO. 2001-521622, which is originally international application of publication No. WO1998/045481) proposed non-thermal devices and methods for amplification of nucleic acids by using denaturant. The devices and methods don't need thermal cycling, and therefore can use thermolabile DNA polymerase. However, the devices and methods need dilution, neutralization or desalting of reaction buffer for every cycle for annealing and polymerizing. In addition, there is inactivation of DNA polymerase by a denaturant in every denaturing step. The number of cycles in pre-determined by channel design and cannot be changed. Precise and complicated liquid handling and a lot of channels are needed.

Ogiwara et al. (Japanese Patent Application Laid-Open No. 2005-6504) also proposed DNA amplification apparatus and method by using denaturant. This apparatus and method can perform DNA amplification at a constant temperature and therefore uses common DNA polymerase. However, this apparatus and method need neutralization of reaction buffer for annealing and polymerizing. Therefore, the volume of the reaction buffer could increase with each cycle. In addition, there is inactivation of DNA polymerase by denaturant in every denaturing step. The number of cycles is pre-determined by channel design and seemingly cannot be changed. Precise and complicated liquid handling and a lot of channels are needed.

### SUMMARY OF THE INVENTION

There is still much room to improve PCR as mentioned above. There is still a great demand to perform PCR in a cheaper, faster, and easier way. Specifically, conventional PCR chips and methods for using these chips proposed before needed thermal cycling and thermostatable DNA polymerases, and therefore are still time-consuming and expensive. Otherwise, denaturants are used instead of thermal cycling, and therefore thermolabile DNA polymerases can be used. However, dilution, neutralization or desalting of reaction buffer is needed for annealing and extension. In addition, there is inactivation of DNA polymerase by denaturants in every denaturing step. The number of cycles is predetermined by channel design and cannot be changed. One aspect of the present invention pertains to providing a method and an apparatus to address these issues.

One aspect of the present invention provides a method and an apparatus that can amplify a nucleic acid in a cheaper, faster and easier way. Specifically, the present invention may provide a method and an apparatus that can amplify a nucleic acid at a substantially constant temperature without special or procedure of dilution, neutralization or desalting. The present invention may also provide a method and an apparatus that can change the number of cycles without special care of changing channel design. The present invention may also provide a method and an apparatus that can amplify a nucleic acid without special procedure of inactivation of a nucleic acid polymerase in every denaturing step.

Other aspect of the present invention may provide a method and an apparatus for amplifying a nucleic acid. Also, the present invention may provide a method and an apparatus for synthesizing a nucleic acid. Yet another aspect of the present invention is a method for forming plural regions containing different concentration of denaturant in a channel.

The present invention may also include, but are not limited to, the followings.

(1) A method for amplifying a nucleic acid, comprising:

(a) forming in a channel an alternating pattern of a region containing a denaturant in an amount sufficient to denature a nucleic acid, and a region containing a denaturant in an amount sufficient to hybridize a denatured nucleic acid with a primer;

(b) exposing a target nucleic acid to the region containing a denaturant in air amount sufficient to denature a nucleic acid, thereby denaturing the target nucleic acid;

(c) exposing the denatured target nucleic acid to the region containing a denaturant in an amount sufficient to hybridize a denatured nucleic acid with a primer, thereby hybridizing the denatured target nucleic acid with a primer;

(d) allowing the primer hybridized with the target nucleic acid in step (c) to be extended using a nucleic acid polymerase;

(e) exposing the extended product obtained in step (d) to a next region containing a denaturant in an amount sufficient to denature a nucleic acid, thereby denaturing the extended product;

(f) exposing the denatured extended product to a next region containing a denaturant in an amount sufficient to hybridize a denatured nucleic acid with a primer, thereby hybridizing the extended product with a primer; and

(g) allowing the primer hybridized with the extended product in step (f) to be extended using a nucleic acid polymerase.

(2) A method according to (1), further comprising repeating the steps (e)-(g) at least once.

(3) A, method according to (1), wherein said region containing a denaturant in an amount sufficient to denature a nucleic acid and said region containing a denaturant in an amount sufficient to hybridize a denatured nucleic acid with a primer are formed by an electrokinetic method.

(4) A method according to (1), wherein said target nucleic acid and said extended product are exposed to the region containing a denaturant in an amount sufficient to denature a nucleic acid and the region containing a denaturant in an amount sufficient to hybridize a denatured nucleic acid with a primer, by an electrokinetic method.

(5) A method according to (1), wherein said region containing a denaturant in an amount sufficient to denature a nucleic acid and said region containing a denaturant in an amount sufficient to hybridize a denatured nucleic acid with a primer are formed by a mechanical method.

(6) A method according to (1), wherein said target nucleic acid and said extended product are exposed to the region containing a denaturant in an amount sufficient to denature a nucleic acid and the region containing a denaturant in an amount sufficient to hybridize a denatured nucleic acid with a primer, by a mechanical method.

(7) A method according to (1), wherein said nucleic acid polymerase is tolerant to the denaturant.

(8) A method according to (3), wherein the denaturant and the nucleic acid polymerase are moved at substantially the same velocity by the electrokinetical method.

(9) A method for amplifying a nucleic acid sequence contained in a nucleic acid, comprising:

(a) providing a first reservoir, a second reservoir, a main channel, a first channel and a second channel disposed all in a fluidic device, wherein the first channel communicating to the first reservoir and the main channel, and the second channel communicating to the second reservoir and the main channel, and at least one to the reservoirs is filled with a liquid containing a denaturant,

(b) forming in the main channel a concentration-cycle region comprising an alternating pattern of a first region having a denaturant of a first denaturant concentration and a second region having the denaturant of a second denaturant concentration, wherein the first denaturant concentration is higher than the second denaturant concentration and the first and second regions being made by introducing the two liquids alternatively or at different flow ratios,

(c) introducing the nucleic acid to the concentration-cycle region,

(d) passing the nucleic acid through the concentration cycle region,

(e) denaturing the nucleic acid in the first denaturant concentration region to produce a denatured nucleic acid,

(f) hybridizing the denatured nucleic acid with a primer in the second denaturant concentration region to produced hybridized nucleic acid, and

(g) extending the primer of the hybridized nucleic acid by a nucleic acid polymerase in the second denaturant concentration region.

(10) The method according to (9), wherein the step of passing the nucleic acid through is performed using electrophoresis by applying a voltage.

(11) The method according to (9), wherein the step of hybridizing the denatured nucleic acid is done without inactivating the denaturant.

(12) The method according to (9), further comprising; (g) introducing the hybridized nucleic acid with the extended primer to the concentration cycle region;

(h) passing the hybridized nucleic acid with the extended primer through the concentration cycle region;

(i) denaturing the hybridized nucleic acid with the extended primer in the first denaturant concentration region to produce a denatured hybridized nucleic acid with the extended primer;

(j) hybridizing the denatured hybridized nucleic acid with the extended primer acid with the primer in the second denaturant concentration region to produce a hybridized nucleic acid; and

(k) extending the primer of the hybridized nucleic acid by the nucleic acid polymerase in the second denaturant concentration region.

(13) The method according to (9), wherein the nucleic acid polymeras is supplied from the first reservoir or the second reservoir.

(14) The method according to (9), wherein, the fluidic device is a microfluidic device.

(15) The method according to (9), wherein at least one of the first and second channels having a pump to control a flow of the channel to form the concentration-cycle region.

(16) The method according to (9), wherein the first, second and main channels communicating at a channel intersection.

(17) The method according to (9), wherein a width of the main channel is in the range of 1 micron to 500 micron.

(18) The method according to (9), wherein the liquid containing the denaturant is supplied by a pump utilizing electrokinetic effect.

(19) The method according to (9), wherein the second denaturant concentration in the range of 0 to 90 percent of the first denaturant concentration.

(20) An apparatus for amplifying a nucleic acid, comprising:

(a) a first reservoir, a second reservoir, a first channel and a second channel, wherein the first reservoir and the second reservoir communicate with the first channel and the second channel, respectively, and at least one of the first and the second reservoirs stores a liquid containing a denaturant;

(b) a main channel communicating with the first channel and the second channel, wherein an alternating pattern of a first region containing a first concentration of a denaturant and a second region containing a second concentration of the denaturants, wherein the first concentration is higher than the second concentration and the first and second regions being made by introducing the two liquids stored in the first and the second reservoir alternatively or at different flow ratios, and wherein the nucleic acid is amplified by exposing the nucleic acid to the alternating pattern of the first region and the second region; and

(c) a sample reservoir and a sample channel, wherein the sample reservoir communicates with the sample channel to the main channel, and the sample reservoir stores a nucleic acid to be amplified.

(21) An apparatus according to (20), further comprising:

a pump introducing the liquid stored in the first and the second reservoirs into the main channel; and

a pump introducing the sample stored in the sample reservoir into the main channel.

(22) A method for synthesizing a nucleic acid, comprising:

(a) forming in a channel an alternating pattern of a region containing a denaturant in an amount sufficient to denature a nucleic acid and a region containing a denaturant in an amount sufficient to hybridize a denatured nucleic acid with a primer;

(b) exposing a target nucleic acid to the region containing a denaturant in an amount sufficient to denature a nucleic acid, thereby denaturing the target nucleic acid;

(c) exposing the denatured target nucleic acid to the region containing a denaturant in an amount sufficient to hybridize a denatured nucleic acid with a primer, thereby hybridizing a primer with the denatured target nucleic acid;

(d) allowing the primer hybridized with the target nucleic acid in step (c) to be extended using a nucleic acid polymerase.

(23) A method according to (1), further comprising a method for synthesizing said target nucleic acid, which comprises:

(a) forming in a channel an alternating pattern of a region containing a denaturant in an amount sufficient to denature a nucleic acid and, a region containing a denaturant in an amount sufficient to hybridize a denatured nucleic acid with a primer;

(b) exposing a pre-target nucleic acid to the region containing a denaturant in an amount sufficient to denature a nucleic acid, thereby denaturing the pre-target nucleic acid;

(c) exposing the denatured pre-target nucleic acid to the region containing a denaturant in an amount sufficient to hybridize a denatured nucleic acid with a primer, thereby hybridizing the denatured pre-target acid with a primer; and

(d) allowing the primer hybridized with the pre-target nucleic acid in step (c) to be extended using a nucleic acid polymerase.

(24) A method according to (1), further comprising a method for synthesizing a target nucleic acid, which comprises:

(a) forming in a channel an alternating pattern of a region containing a denaturant in an amount sufficient to denature a nucleic acid and a region containing a denaturant in an amount sufficient to hybridize a denatured nucleic acid with a primer;

(b) exposing a pre-target nucleic acid to the region containing a denaturant in an amount sufficient to denature a nucleic acid, thereby denaturing the pre-target nucleic acid;

(c) exposing the denatured pre-target nucleic acid to the region containing a denaturant in an amount sufficient to hybridize a denatured nucleic acid with a primer, thereby hybridizing the denatured pre-target acid with a primer; and

(d) allowing the primer hybridized with the pre-target nucleic acid in step (c) to be extended using a nucleic acid polymerase.

Also, the invention provides a method for amplifying a target nucleic acid, which comprises (a) a step of synthesizing a target nucleic acid from a pre-target nucleic acid, and (b) a step of amplifying a target nucleic acid, said step (a) of synthesizing a target nucleic acid comprising the following steps of (i) to (iv):

(i) forming in a channel an alternating pattern of a region containing a denaturant in an amount sufficient to denature a nucleic acid and a region containing a denaturant in an amount sufficient to hybridize a denatured nucleic acid with a primer;

(ii) exposing a pre-target nucleic acid to the region containing a denaturant in an amount sufficient to denature a nucleic acid, thereby denaturing the pre-target nucleic acid;

(iii) exposing the denatured pre-target nucleic acid to the region containing a denaturant in an amount sufficient to hybridize a denatured nucleic acid with a primer, thereby hybridizing the denatured pre-target acid with a primer;

(iv) allowing the primer hybridized with the pre-target nucleic acid in step (iii) to be extended using a nucleic acid polymerase thereby securing a target nucleic acid; and

said step (b) of amplifying a target nucleic acid comprising the following steps of (v) to (xi):

(v) providing a first reservoir, a second reservoir, a main channel, a first channel and a second channel disposed all in a fluidic device, wherein the first channel communicating to the first reservoir and the main channel, and the second channel communicating to the second reservoir and the main channel, and at least one of the reservoirs is filled with a liquid containing a denaturant;

(vi) forming in the main channel a concentration-cycle region comprising an alternating pattern of a first region having a denaturant of a first denaturant concentration and a second region having the denaturant of a second denaturant concentration, wherein the first denaturant concentration is higher than the second denaturant concentration and the first and second regions being made by introducing the two liquids alternatively or at different flow ratios;

(vii) introducing the target nucleic acid to the concentration-cycle region;

(viii) passing the target nucleic acid through the concentration cycle region;

(ix) denaturing the target nucleic acid in the first denaturant concentration region to produce a denatured nucleic acid;

(x) hybridizing the denatured target nucleic acid with a primer in the second denaturant concentration region to produce a hybridized nucleic acid; and

(xi) extending the primer of the hybridized target nucleic acid by a nucleic acid polymerase in the second denaturant concentration region.

Further scope of applicability of the present invention will become apparent from the detailed description given hereinafter.

### BRIEF DESCRIPTION OF THE DRAWINGS

The present invention will become more fully understood from the detailed description given hereinbelow and the accompanying drawings which are given by way of illustration only, and thus are not limitative of the present invention.

FIG. 1 is a schematic view showing an example of the apparatus of the present invention.

FIG. 2 is a schematic view showing another example of the apparatus of the present invention.

FIG. 3 is a schematic view showing another example of the apparatus of the present invention.

FIG. 4 is a schematic view showing another example of the apparatus of the present invention.

FIG. 5 is a schematic view showing another example of the apparatus of the present invention.

### EXPLANATIONS OF LETTERS AND NUMERALS IN FIGS

1: part for forming concentration-cycle region
2: concentration-cycle channel
3: sample part
4: denaturant buffer reservoir
5: buffer reservoir
6: denaturant buffer channel
7: buffer channel
8: sample reservoir
9: sample injection channel
10: waste reservoir
11: sample waste reservoir
12: sample injection region

### DETAILED DESCRIPTION OF THE INVENTION

The amplification reaction to be used in the present invention included PCR and other well-known amplification reaction in the art.

The nucleic acid to be used in the present invention includes double-stranded DNA, single-stranded DNA, double-stranded RNA, single-stranded RNA, and double-stranded DNA-RNA hybrid. As used herein, the term “target nucleic acid” is defined as an initial nucleic acid containing a specific nucleic acid sequence to be amplified. The target nucleic acid can be obtained from biological samples such as blood, cells, foods, or environmental samples (e.g., soil, river water, seawater, activated sludge, or methane fermentation sludge).

The primer to be used in the present invention can be prepared by methods that are well known in the art. The primer is sufficiently complementary to the sequence of the strand to which the primer hybridizes, which means the primer is not exactly complementary to the sequence of the strand to which the primer hybridizes. In one embodiment of the present invention, the primer is contained in a solution containing a sample, hereinafter we call this solution as a “sample solution”. In another embodiment of the present invention, the primer is contained in all solutions or buffers.

The nucleic acid polymerase, to be used in the present invention includes E. coli DNA polymerase such as E. coli DNA polymerase I and Klenow fragment of E. coli DNA polymerase I; T4 DNA polymerase; T7 DNA polymerase; reverse transcriptase; and RNA polymerase such as RNA replicase. Also, thermostable polymerases such as Taq polymerase, Pfu DNA polymerase and KOD DNA polymerase can be used in the present invention. In one embodiment, thermostable polymerase is used. It is, of course, within the scope of the invention to use mixtures of different nucleic acid polymerases, if desired. It is also within the scope of the invention to use mixtures of different types of nucleic acid polymerases, if desired.

The denaturant to be used in the present invention includes 1) chaotropic agents, for example formamide, urea and guanidinium hydrochloride and 2) the mixture of chaotropic agents. Bases raising pH and acids decreasing pH can also be used as the denaturant. In addition, an agent inhibiting hydrogen bond between nucleic acids can be used as the denaturant of the present invention. The denaturant of the present invention can carry a positive or negative charge, or can be electrically neutral.

The channel to be used in the present invention includes capillaries and microchannels formed on a microchip. The diameter of capillary and the width and depth of microchannel are preferably from 1 micrometer to 500 micrometers, more preferably from 10 micrometers to 100 micrometers. The shape of microchannel includes, but is not limited to, rectangular, triangular, trapezoid, and circular. In the present invention, the channel in which the concentration-cycle region is formed is referred to as a concentration-cycle channel or a main channel.

The microchip is typically composed of two substrates, but can also be composed one substrate. If the microchip is composed of two substrates, channels are formed in one substrate by using a microfabrication technology and holes for reservoirs are created in another substrate by use of machining such as drilling and punching. These two substrates are bonded by a bonding technology, which makes the microchip with the channels and reservoirs at predetermined positions. As is well known to those skilled in the art, the microchip of the present invention is also referred to as a microfluidic device, a microchip laboratory, and the microchip of the present invention can be referred to as a microfluidic device, a microchip laboratory and the like.

The material to be used for making the microchip includes, but is not limited to, a polymer such as silicone resin (e.g., poly(dimethylsiloxane)); acrylic resin (e.g., poly(methyl methacrylate)); polycarbonate; polyetheretherketone; and cyclic olefin copolymer. Glass, silica glass and silicon are also used as materials for making microchips.

The microfabrication technology to form channels in polymer materials includes replication methods such as hot embossing, injection molding casting, and soft lithography, and direct fabrication methods such as laser ablation, plasma etching, X-ray lithography, LIGA, layering, and end milling. The bonding technology to be used for polymer materials includes thermal bonding, adhesive bonding, ultrasonic welding, oxygen plasma, and lamination.

The microfabrication technology to form channels in glass, silica glass and silicon materials includes photolithography. The channels are created by wet etching such as isotropic etching, anisotropic etching and electrochemical etching; and dry etching such as plasma etching and reactive ion etching. The bonding technology to be used for glass, silica glass and silicon materials includes anodic bonding, thermal bonding and HF bonding.

As used herein, the electrokinetic pump is defined as pumps utilizing the motion of substance such as molecule and particle in applied electric field. The electrokinetic pump to be used in the present invention includes electroosmotic flow, electrophoresis, dielectrophoresis, and the combination of them. On the other hand, the mechanical pump is defined as pumps with moving parts. The mechanical pump to be used in the present invention includes a syringe pump, a diaphragm pump, a peristaltic pump, a gear pump, and a turbo pump.

The “term substantially same velocity” means that the difference of the velocities is acceptable to a certain extent. Particularly, the difference of the velocities is acceptable to the extent that some of the nucleic acid polymerase don't contact to the next higher denaturant regions and remain its activity in the region where the primer extension occurs until the target nucleic acid and extended products go though the region, or pass through the region. The acceptable velocity of the nucleic acid polymerase is within 5% of the velocity of the denaturant. However, it should be noted that the acceptable difference between the velocities depends on various conditions such as the length of high and low-concentration regions; the number of cycle; the times needed for denaturing hybridizing and extension; the electrophoretic velocity of the target nucleic acid, the extended products, the nucleic acid polymerase, and the denaturant; the velocity of electroosmotic flow; and the velocity of mechanical pumping. The typical electrophoretic velocity of target nucleic acid and extended product in the direction of anode is from 10 micron/second to 1 mm/second. The typical velocity of electroosmotic flow in the direction of cathode is from 10 micron/second to 1 mm/second. The typical velocity of denaturant in the direction of cathode is from 10 micron/second to 1 mm/second when urea and formamide are used as denaturant. For instance, in the case that the length of low-concentration regions is 9 mm, the number of cycle is 30, the electrophoretic velocity of the target nucleic acid and extended product is 300 micron/second in the direction of anode, and the velocity of the denaturant and electroosmotic flow is 200 micron/second in the direction of cathode, the acceptable velocity of the nucleic acid polymerase is within 5% of the velocity of the denaturant.

The term “tolerant to a denaturant” used in the present invention means that a substance can maintain its activity in a solution containing at denaturant or that a substance can recover its activity after being exposed to a denaturant. The term “containing denaturant in an amount sufficient to hybridize” is identical to the term “containing denaturant in a sufficiently low amount to permit hybridization” in the present invention.

As used herein, the term “pre-target nucleic acid” refers to a nucleic acid used as a template for the synthesis of the target nucleic acid which is used for amplifying a specific nucleic acid sequence.

The present invention can amplify at least one specific nucleic acid sequence present in at least one target nucleic acid. The target nucleic acid might be typically double-stranded. In a preferred embodiment, the present invention is performed in a channel on a microchip.

In the present invention, amplification is performed in a “concentration-cycle region” formed in a channel. As used herein the term “concentration-cycle region” refers to a region which comprises an alternating pattern of alt least two regions containing different concentrations of denaturant; namely (a) a region containing denaturant at higher concentration (“high-concentration region”) and (b) a region containing denaturant at lower concentration (“low-concentration region”). In the present invention, a region consisting of one high-concentration region and one low-concentration region is defined as one “cycle”. Namely, the concentration-cycle region of the present invention comprises one or more cycles. Further, each of at least two regions containing different concentration of denaturant is referred to as a first, second, third region, respectively. In one embodiment, the concentration of denaturant in the first region is higher than that in the second region. The concentration of the second region is preferably in the range of 0 to 90 percent of that of the first region, and more preferably in the range of 0 to 70, and most preferably in the range of 0 to 50.

In one embodiment of the invention, the cycle comprises only two regions containing different concentrations of denaturant (i.e. a low-concentration region and a high-concentration region). In this embodiment, the step of denaturing a nucleic acid can be performed in the high-concentration region, and the steps of hybridizing and extending primers can be performed in the low-concentration region.

In another embodiment, the cycle comprises three regions containing different concentrations of denaturant, that is, low-concentration region, medium-concentration region and low-concentration region. In this embodiment, the denaturing step can be performed in the high-concentration region, and the hybridizing step and the extending step can be performed in the low-concentration region and the medium-concentration region, respectively.

In the present invention, every cycle doesn't have to contain same pattern of high- and low-concentration regions. The length of each cycle can be changed. The lengths of the high- and low-concentration regions can be different in each cycle. The concentrations of denaturant in the high- and low-concentration regions can be different in each cycle. In particular, lowering a concentration of denaturant in a low-concentration region in certain cycles can reduce undesirable nonspecific products.

The appropriate condition including denaturant concentration of the higher denaturant concentration region and temperature can be decided based on various properties of the amplification product, the target nucleic acid, the primer, the nucleic acid polymerase and so on. For example, appropriate condition can be determined depending on Tm and GC contents of the nucleic acid and the like.

In one aspect, the present invention provides a method for amplifying a nucleic acid, comprising:

(a) forming in a channel an alternating pattern of a region containing a denaturant in an amount sufficient to denature a nucleic acid, and a region containing a denaturant in an amount sufficient to hybridize a denatured nucleic acid with a primer;

(b) exposing a target nucleic acid to the region containing a denaturant in an amount sufficient to denature a nucleic acid, thereby denaturing the target nucleic acid;

(c) exposing the denatured target nucleic acid to the region containing a denaturant in an amount sufficient to hybridize a denatured nucleic acid with a primer, thereby hybridizing the denatured target nucleic acid with a primer;

(d) allowing the primer hybridized with the target nucleic acid in step (c) to be extended using a nucleic acid polymerase;

(e) exposing the extended product obtained in step (d) to a next region containing a denaturant in an amount sufficient to denature a nucleic acid, thereby denaturing the extended product;

(f) exposing the denatured extended product to a next region containing a denaturant in an amount sufficient to hybridize a denatured nucleic acid, with a primer thereby hybridizing the extended product with a primer;

(g) allowing the primer hybridized with the extended product in step (f) to be extended using a nucleic acid polymerase; and

(h) optionally, repeating steps (e)-(g) at least once.

In this embodiments the high-concentration region corresponds to “a region containing a denaturant in an amount sufficient to denature a nucleic acid”, and the low-concentration region corresponds to “a region containing a denaturant in an amount sufficient to hybridize a denatured nucleic acid with a primer”.

In the present invention, a nucleic acid sequence is amplified as described below. To begin with, concentration-cycle region is formed in a channel. Then, the target nucleic acid is allowed to go through the concentration-cycle region, thereby amplifying the nucleic acid sequence.

In the present invention, the target nucleic acid is exposed to the initial region containing a denaturant in an amount sufficient to denature a nucleic acid, meaning an initial high-concentration region, thereby denaturing the target nucleic acid into two single-stranded nucleic acids. Then, the denatured target nucleic acids are exposed to the initial region containing a denaturant in an amount sufficient to hybridize a denatured nucleic acid with a primer, meaning an initial low-concentration region, thereby hybridizing the denatured two single-stranded nucleic acid with primers. In a typical embodiment, the number of primers is two. Then, each of the primers is extended to form a double-stranded nucleic acid using a nucleic acid polymerase. Each of the two primers is designed to be sufficiently complementary to the sequence of each of the two single-stranded nucleic acids, and therefore each of the two primers can hybridizes to each of the two single-stranded, nucleic acids. In the next high-concentration region, the extended products generated in the initial cycle are denatured into two single-stranded nucleic acids. In the next low-concentration region, each of the two primers hybridizes to each of the two single-stranded nucleic acids. Then, each of the two primers is extended to form double-stranded nucleic acids.

In the present invention, steps of denaturing, hybridizing and extending are automatically repeated while the extended products go through the concentration-cycle region, or pass through the concentration-cycle channel. In this way, a specific nucleic acid sequence between the two regions where the two primers hybridize is amplified while the nucleic acid goes through, or passes through at least two cycle formed in a channel. In one embodiment of the present invention, while the nucleic acid passes through, or goes through, the concentration-cycle region comprising plural cycles is formed in a channel, each of the denaturing step, the hybridizing step and the extending step are repeated. In one embodiment, the number of cycles comprised in the concentration-cycle region can be from 2 to 50. The number of repetition of cycles can be determined so as to secure an effective improvement of recovery rate of amplification.

In one embodiment of the invention, time required for the target nucleic acid and extended products to go through one cycle can be from 200 milliseconds to 40 min, more preferably from 2 second to 6 min, much more preferably from 2 seconds to 2 min. The time required for denaturing a target nucleic acid and extended products can be from 100 millisecond to 10 min, more preferably from 1 second to 3 min, much more preferably from 1 second to 1 min. The time required for extending primers can be from 100 milliseconds to 30 min, more preferably, from 1 second to 3 min, much more preferably from 1 second to 1 min. However, it should be rioted that the time required for denaturing a target nucleic acid and extended products depends on various factors such as length of nucleic acid, temperature, concentration of denaturant in high-concentration region and ion concentration. In addition, it should be noted that the time required for extending primers depends on activity of the nucleic acid polymerase to be used.

In one embodiment of the invention, the length of a high-concentration region can be from 10 micron to 10 cm, more preferably from 100 micron to 5 cm, much more preferably from 1 mm to 3 cm. However, it should be noted that the length of a high-concentration region depends on various factors such as mobility and length of nucleic acid, temperature, and ion concentration. The length of a low-concentration region can be from 10 micron to 10 cm, more preferably from 100 micron to 5 cm, much more preferably from 1 mm to 3 cm. However, it should be noted that the length of a low-concentration region depends on various factors such as mobility and length of nucleic acid, temperature, and activity of the nucleic acid polymerase to be used.

In the present invention, it is preferable to form a high-concentration region before the initial cycle in order to ensure denaturing of the target nucleic acid. After the last cycle, it is preferable to form a region containing denaturant in an amount sufficient to ensure extension of primers.

In another embodiment, the target nucleic acid is single-stranded nucleic acid. In this embodiment, one of the two primers hybridizes to the single-stranded target nucleic acid having substantially complimentary sequence to the primer, and, then, the primer is extended to form a double-stranded nucleic acid in the initial cycle.

In yet another embodiment, the synthesized double-stranded nucleic acid can be used as the target nucleic acids and a specific sequence of the nucleic acid, to which the two primers can hybridize, is amplified in the same manner as described above.

In one embodiment, the present invention can amplify plural parts of specific sequences in plural target nucleic acids. By using one or more different pairs of primers which can hybridize with different sequences of the target nucleic acids in this reaction, plural parts of sequences can be amplified. The present invention can also amplify plural specific sequences in a target nucleic acid.

The present invention has many advantages. Since a nucleic acid is denatured using denaturants instead of heating, amplification reaction can be performed at a relatively low temperature. This means that time-consuming thermal cycling and expensive thermostable polymerases are not needed in the amplification reaction of the present invention, which makes the amplification reaction faster and cheaper. In addition, in the present invention, the target nucleic acid and extended product sequentially go through the high- and lows-concentration regions, resulting in passing through plurality of the high-concentration regions and plurality of low-concentration regions respectively. Therefore, the extending step can be performed without dilution, neutralization, and desalination, which makes the amplification reaction easier. In addition, the target nucleic acid doesn't contact nucleic acid polymerase until amplification starts, which makes it possible to reduce nonspecific amplification without using bothering hot-start techniques or expensive automatic hot-start polymerases.

In the present invention, samples and reagents such as denaturants, primers and nucleic acid polymerases can be transported using any commonly-used method. In one embodiment, samples and reagents are transported by an electrokinetic method and/or a mechanical method.

In one embodiment of the present invention, the concentration-cycle region comprising at least two high-concentration region and at least two low-concentration region is formed by an electrokinetic pump which utilizes electrokinetic effect. If denaturant is electrically neutral, the denaturant can be transported by electroosmotic flow. If the denaturant is electrically positive or negative, the denaturant can be transported not only by electroosmotic flow but also electrophoresis.

In the present invention, the electrokinetic pump is preferred since the pump has the following advantages. The electrokinetic pump can be performed simply by applying voltages. There is no occurrence of pulsation. The pump can make a flow with a flat profile, or so-called “plug flow”, in a channel. These aspects of electrokinetic pump offer precise flow control and ease of operation. Also, these aspects of the electrokinetic pump are preferable for forming the concentration-cycle region in a microchannel. In addition, the electrokinetic pump also offers ease of parallelization because the only thing for parallelization is increasing electrodes which makes high-throughput amplification easier.

In another embodiment, the target nucleic acid and the extended product are exposed to the high-concentration region and the low-concentration region by an electrokinetic method. The target nucleic acid and the extended product typically have negative charges. Therefore, if there is electroosmotic flow, the target nucleic acid and the extended product are transported by a sum of electroosmotic flow and electrophoresis. If there is no electroosmotic flow, the target nucleic acid and the extended product are transported by electrophoresis.

There are many advantages of pumping electrokinetically. The electrokinetic pump can be performed simply by applying voltages. There is no occurrence of pulsation. It has a flat profile, or so-called plug flow. These aspects offer precise flow control and ease of operation, which make it easy to precisely control volume of a sample containing the target nucleic acid, and therefore perform a reproducible amplification. In addition, if the denaturant is electrically neutral, the negative-charged target nucleic acid and the extended product close to the concentration-cycle region are transported by themselves even though there is electroosmotic flow.

In another preferred embodiment, the mechanical pump is used since the mechanical pump can easily transport solutions that are difficult to be transport by the electrokinetic pump. In particular, the mechanical pump can be preferably used for transporting highly ionic solutions, different conductive solutions, and different pH solutions. Thus, the mechanical pump is preferred for a high or low pH solution used as denaturant and for the target nucleic acid in highly ionic solutions.

One aspect of the present invention provides a method of forming the concentration-cycle region. In a preferred embodiment, the concentration-cycle region is formed by electrokinetic method. Another aspect of the present invention provides a method of exposing the target nucleic acid and extended products to the concentration-cycle, region.

In the present invention, the concentration-cycle region can be formed by mixing at least two buffers, each of which contains different concentration of denaturant. For example, the concentration-cycle region can be formed by mixing a buffer containing denaturant with a buffer not containing a denaturant. In another embodiment, the concentration-cycle region can be formed by mixing a buffer containing higher concentration of a denaturant with a buffer containing lower concentration of a denaturant.

As used herein, in the case that formamide and urea are used as the denaturant the buffer containing 40% (v/v) of formamide and 7M of urea is defined as “100%” denaturant buffer. A buffer containing no denaturant is defined as “0%” denaturant buffer. For example, “50%” denaturant buffer can be prepared by mixing “100%” denaturant buffer and “0%” denaturant buffer at the ratio of 1/1.

The typical denaturant concentration of the high-concentration region is more than “50%”, more preferably more than “90%” when the amplification reaction is performed at 50° C. The denaturant concentration of the high-concentration region can be more than “100%”. The appropriate condition including denaturant concentration of the higher denaturant concentration region and temperature can be decided based on various properties of the amplification product, the target nucleic acid, the primer, the nucleic acid polymerase and so on. The typical denaturant concentration of the low-concentration region is from “0%” to “50%”. The appropriate condition including denaturant concentration of the lower denaturant concentration and temperature can be decided based on various properties of the amplification product, the target nucleic acid, the primer, the nucleic acid polymerase and so on.

In a preferred embodiment, the nucleic acid polymerase is tolerant to denaturant. In the present invention, if the velocity of the polymerase is different from the velocity of the denaturant in a channel, it is possible that the polymerase is exposed to the denaturant. In that case, if the polymerase was not tolerant to the denaturant, the polymerase would lose its activity. However, if the polymerase was tolerant to the denaturant, it would maintain its activity even though it is exposed the denaturant. In addition, there is a possibility that, around an interface between high-concentration regions and low-concentration regions in the concentration-cycle region, the denaturant in the high-concentration region diffuses into the low-concentration region and comes into contact with the polymerase. In such a case, the pool erase is preferably tolerant to the denaturant.

In a preferred embodiment, the denaturant and the nucleic acid polymerase are moved at substantially the same velocity. If the velocity of the denaturant contained in one region is the same as the velocity of the polymerase contained in a next region, the polymerase is less likely to be mixed with the denaturant. Thus, ere if the polymerase is not tolerant to a denaturant, roving the polymerase at substantially the same velocity as the denaturant enables the polymerase to maintain its activity for longer period of time during the amplification reaction.

If the denaturant is electrically neutral, it is preferable that the amplification reaction is performed under the condition that pH of the buffer to contain the polymerase is the same as the isoelectric point of the polymerase. In this embodiment, the denaturant and the nucleic acid polymerase can be transported at substantially the same velocity by an electrokinetic pump.

In one embodiment of the present invention, a polymerase can be contained in all solutions and buffers used in the invention. If an electrokinetic pump is used for transporting solutions or buffers in the microchip, a nucleic acid polymerase being electrically neutral is contained preferably in buffers in the denaturant reservoir and/or buffer reservoir, and more preferably, in a buffer in the buffer reservoir. If a polymerase has a positive charge, it is preferred that the polymerase is contained in buffers in denaturant reservoir and/or buffer reservoir. If a polymerase has a negative charge, it is preferred that the polymerase is contained in a sample solution and buffers in reservoirs other than denaturant and buffer reservoirs.

In one aspect, the present invention is a method for amplifying a nucleic acid sequence contained in a nucleic acid, comprising:

(a) providing a first reservoir, a second reservoir, a main channel, a first channel and a second channel disposed all in a fluidic device, wherein the first channel communicating to the first reservoir and the main channel, and the second channel communicating to the second reservoir and the main channel, and at least one of the reservoirs is filled with a liquid containing a denaturant,

(b) forming in the main channel a concentration-cycle region comprising an alternating pattern of a first region having a denaturant of a first denaturant concentration and a second region having the denaturant of a second denaturant concentration, wherein the first denaturant concentration is higher than the second denaturant concentration and the first and second regions being made by introducing the two liquids alternatively or at different flow ratios,

(c) introducing tire nucleic acid to the concentration-cycle region,

(d) passing the nucleic acid through the concentration cycle,

(e) denaturing the nucleic acid in the first denaturant concentration region to produce a denatured nucleic acid,

(f) hybridizing the denatured nucleic acid with a primer in the second denaturant concentration region to produce a hybridized nucleic acid, and

(g) extending the primer of the hybridized nucleic acid by a nucleic acid polymerase in the second denaturant concentration region.

In one aspect the present invention provides an apparatus for amplifying a nucleic acid, comprising:

(a) a unit pumping a denaturant, wherein the unit combines at least two solutions containing the denaturant at different concentration to form in a channel an alternating pattern of a region containing a denaturant in an amount sufficient to denature the nucleic acid and a region containing a denaturant in an amount sufficient to hybridize the denatured nucleic acid with a primer;

(b) a channel, wherein the regions containing the denaturant at different concentrations are formed; and

(c) a unit pumping a target nucleic acid, which introduces the target nucleic acid into the channel.

The unit pumping a denaturant forms the concentration-cycle region comprising high- and low-concentration regions cyclically by mixing at least two buffers containing different concentrations of denaturant. The concentration-cycle region is injected into the concentration-cycle channel, or the main channel.

The unit pumping a target nucleic acid introduces the target nucleic acid into the concentration-cycle channel.

This apparatus can change the pattern and/or the number of cycles in the concentration-cycle region by changing the mixing pattern of at least two buffers containing different concentrations of denaturant. Thus, according to the present invention, amplification of nucleic acid car, be conducted without changing channel arrangement or channel arrangement on a chip. In addition, this apparatus can easily control the reaction times of denaturing, hybridizing and extending without changing channel design or channel arrangement by controlling the lengths of the high- and low-concentration regions. These aspects of the present invention make it possible to conduct amplification reaction flexibly according to the target nucleic acid and the product of amplification.

In a preferred embodiment, the part for forming a concentration-cycle region comprises: at least two buffer reservoirs to be filled with buffers containing different concentrations of denaturant; and at least two buffer channels connected to the reservoirs, respectively. The two buffer channels converge on the concentration-cycle channel. In one embodiment, at least two buffer channels can communicate with the concentration-cycle channel at an intersection. In another embodiment, at least two buffer channels can communicate with the concentration-cycle channel at two or more intersections. The buffer channels can have one or more pumps to control a flow in the channels to form the concentration-cycle region in the concentration-cycle channel.

The concentration-cycle channel is connected to the sample part. The sample part comprises sample reservoir to be filled with a sample containing the target nucleic acid. The specific nucleic acid sequence in injected target nucleic acid is amplified in the concentration-cycle channel. In one embodiment, the nucleic acid ran be transported relatively against the first and second denaturants using electrophoresis occurred by applying a voltage between the main channel.

In one embodiment, the unit for pumping a denaturant has two reservoirs for buffer containing different concentrations of denaturant. In one embodiment, the buffer in one reservoir does not contain denaturant, and the buffer in the other contains denaturant.

In one aspect of, the present invention is an apparatus for amplifying a nucleic acid, comprising:

(a) a first reservoir, a second reservoir, a first channel and a second channel, wherein the first reservoir and the second reservoir communicate with the first channel and the second channel, respectively, and at least one of the first and the second reservoirs stores a liquid containing a denaturant;

(b) a main channel communicating with the first channel and the second channel wherein an alternating pattern of a first region containing a first concentration of a denaturant and a second region containing a second concentration of the denaturant, wherein the first concentration is higher than the second concentration and the first and second regions being made by introducing the two liquids stored in the first and the second reservoir alternatively or at different flow ratios, and wherein the nucleic acid is amplified by exposing the nucleic acid to the alternating pattern of the first region and the second region; and

(c) a sample reservoir and a sample channel, wherein the sample reservoir communicates with the sample channel to the main channel, and the sample reservoir stores a nucleic acid to be amplified.

In a preferred embodiment, the apparatus of the present invention further comprising: a pump introducing the liquid stored in the first and the second reservoirs into the main channel; and a pump introducing the sample stored in the sample reservoir into the main channel. As described above, samples and reagents such as denaturants, primers and nucleic acid polymerases can be transported using any commonly-used method. In one embodiment, samples and regents are transported by one or more pumps which utilize electrokinetic effect and/or mechanical effect. In the present invention, the electrokinetic pump is preferred.

Another aspect of the present invention provides a method for synthesizing a nucleic acid, comprising:

(a) forming in a channel an alternating pattern of a region containing a denaturant in an amount sufficient to denature a nucleic acid and a region containing a denaturant in an amount sufficient to hybridize a denatured nucleic acid with a primer;

(b) exposing a target nucleic acid to the region containing a denaturant in an amount sufficient to denature a nucleic acid, thereby denaturing the target nucleic acid;

(c) exposing the denatured target nucleic acid to the region containing a denaturant in an amount sufficient to hybridize a denatured nucleic acid with a primer, thereby hybridizing a primer with the denatured target nucleic acid; and

(d) allowing the primer hybridized with the target nucleic acid in step (c) to be extended using a nucleic acid polymerase.

The present invention also provides a method for synthesizing at least a specific nucleic acid sequence. This method is preferable for cycle sequencing reaction and reverse-transcription reaction. The synthesis is performed in a channel where a concentration-cycle region is formed. The concentration-cycle region cyclically contains at least one cycle.

In the present invention, a specific nucleic acid sequence is synthesized as follows. In a typical embodiment, the target nucleic acid is double stranded, and the number of primers is one. The target nucleic acid is denatured into two single-stranded nucleic acids in the high-concentration region. The primer hybridizes to one of the two single-stranded nucleic acids in the low-concentration region, and is extended to form a double-stranded nucleic acid by a nucleic acid polymerase. The double-stranded nucleic acid generated in the previous cycle is denatured into two single-stranded nucleic acid in the next high-concentration region. The primer hybridizes to one of the two singles-stranded nucleic acids in the next low-concentration region, and is extended to make another double-stranded nucleic acid. Thus, the product of this reaction is the single-stranded nucleic acids that have the primer sequence at one end.

In another embodiment, the target nucleic acid is a single-stranded nucleic acid. The number of cycle is at least one.

Another aspect of the present invention provides a method for synthesizing a target nucleic acid to be amplified subsequently, which comprises:

(a) forming in a channel an alternating pattern of a region containing a denaturant in an amount sufficient to denature a nucleic acid and a region containing a denaturant in an amount sufficient to hybridize a denatured nucleic acid with a primer;

(b) exposing a pre-target nucleic acid to the region containing a denaturant in an amount sufficient to denature a nucleic acid, thereby denaturing the pre-target nucleic acid;

(c) exposing the denatured pre-target nucleic acid to the region containing a denaturant in an amount sufficient to hybridize a denatured nucleic acid with a primer, thereby hybridizing the denatured pre-target acid with a primer; and

(d) allowing the primer hybridized with the pre-target nucleic acid in step (c) to be extended using a nucleic acid polymerase.

The present invention also provides a method for synthesizing the target nucleic acid that is used for amplifying a specific nucleic acid sequence. This method is preferable for reverse-transcription PCR (RT-PCR). The synthesis is performed in a channel where a concentration-cycle region is formed. The concentration-cycle region cyclically contains at least one cycle.

The target nucleic acid is synthesized as follows. In a typical embodiment, the pre-target nucleic acid is single stranded. The typical number of primers is two. The typical number of cycles comprised in the concentration-cycle region is one. The pre-target nucleic acid is denatured in the high-concentration region. One of the two primers hybridized to the single-stranded pre-target nucleic acid in the low-concentration region, and is extended by a nucleic acid polymerase to form a double-stranded nucleic acid which can be used as a, target nucleic acid. In another embodiment, the pre-target nucleic acid is a double-stranded nucleic acid. The number of cycle is at least one.

In yet another embodiment, the synthesized double-stranded nucleic acid is used as the target nucleic acid for amplifying a specific nucleic acid sequence. The procedure for amplification is the same as describe above.

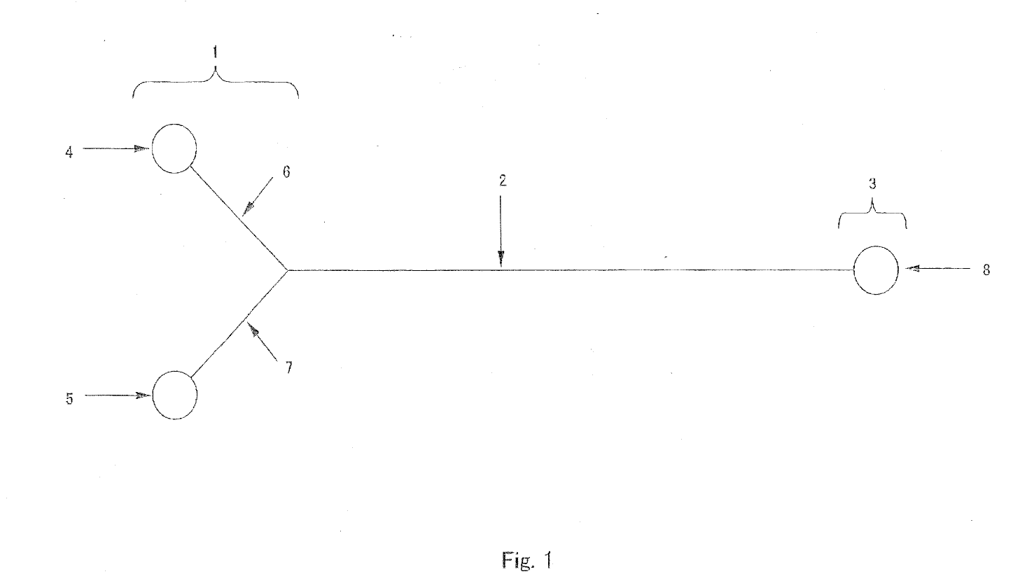

FIG. 1 shows an example of an apparatus of the present invention. This apparatus is suitable for a case in which the target nucleic acid moves in the opposite direction to the flow of the denaturant. The apparatus comprises a part for forming concentration-cycle region 1, a concentration-cycle channel 2, and a sample part 3. The concentration cycling part 1 comprises the denaturant buffer reservoir 4, the buffer reservoir 5, the denaturant buffer channel 6, and the buffer channel 7. The sample part comprises the sample reservoir 8. The denaturant buffer channel 6 and the buffer channel 7 converge in the concentration-cycle channel 2 at an intersection. The concentration-cycle channel 2 is connected to the sample reservoir 8.

As shown in FIG. 1, there is no injection tool or special procedure for injecting desalting chemicals, which functions as an agent for inactivating a denaturant, into the concentration cycle. Therefore, the step of hybridizing the denatured nucleic acid is done without inactivating the denaturant.

In an operation for amplifying a specific nucleic acid sequence, the denaturant buffer reservoir 4 is filled with a buffer containing denaturant, and the buffer reservoir 5 is filled with a buffer containing no denaturant. The sample reservoir 8 is filled with a sample solution containing a target nucleic acid. Each of three electrodes is inserted into the denaturant buffer reservoir 4, the buffer reservoir 5, and the sample reservoir 8 respectively. By changing the electric potentials applied to the reservoir 4 and the reservoir 5, a concentration-cycle region comprising high- and low-concentration regions is formed. On the other hand, the sample reservoir is grounded. The high-concentration region in a cycle is made by injecting the buffer containing denaturant into the concentration-cycle channel 2 from the denaturant buffer reservoir 4 through the denaturant buffer channel 6. The low-concentration region in a cycle is made by injecting the buffer containing no denaturant into the concentration-cycle channel 2 from the buffer reservoir 5 through the buffer channel 7. The concentration-cycle region is made by repeating injection of the buffer containing denaturant and the buffer containing no denaturant or substantially less denaturant, alternatively. The target nucleic acid is injected into the concentration-cycle channel 2 from the sample reservoir 8. The injected target nucleic acid goes through the concentration-cycle region in the concentration-cycle channel 2, or passes through the concentration-cycle region, and is amplified in the concentration-cycle channel 2. Also, the volume of the reaction liquid existing in the concentration-cycle channel 2 can be kept in substantially constant and does not increase in accordance with the progress of the reactions, and therefore, there are no need for adjusting the amount of the injection of reagents in accordance with the amplification. The amplification product is detected at a point in the channels or collected from the reservoir 4 and the reservoir 5. It is also possible to monitor all of the channels, which is preferable for real-time and quantitative amplification techniques it is also possible to conduct repetitive amplification. That is, after introducing the hybridized nucleic acid with the extended primer to a concentration-cycle region in the concentration-cycle channel 2 after the first amplification of a nucleic acid so as to pass the hybridized nucleic acid with the extended primer through the concentration cycle region, following steps are needed—(1) to denature the hybridized nucleic acid with, the extended primer in the first denaturant concentration region to produce a denatured hybridized nucleic acid with the extended primer, (2) to hybridize the denatured hybridized nucleic acid with the extended primer acid with the primer in the second denaturant concentration region to produce a hybridized nucleic acid, and (3) to extend the primer of the hybridized nucleic acid by the nucleic acid polymerase, in the second denaturant concentration region.

Appropriate electric potentials to form a concentration-cycle region depend on channel design or channel arrangement and properties of buffers. The lengths of the high- and low-concentration regions and the cycle number are determined by reaction temperature and various properties of an amplification product, a target nucleic acid a primer and a nucleic acid polymerase. The requisite components for amplification such as a primer, a nucleic acid polymerase, MgCl₂, and KCl are allowed to be present at each reservoir and channel. It is preferable that the nucleic acid polymerase is not present in the sample reservoir 8 to suppress nonspecific amplification. It is also preferable that the nucleic acid polymerase is not present in the denaturant buffer reservoir 5 to decrease the consumption of the nucleic acid polymerase.

It is also possible to form a region containing denaturant at a different concentration from the concentration of the denaturant buffers filled in the denaturant buffer reservoir 4 and the buffer reservoir 5 by means of mixing the two buffers at appropriate ratios. For example, in the case that the reservoir 4 is filled with “100%” denaturant buffer and the reservoir 5 is filled with “0%” denaturant buffer, a region containing denaturant at “80%” in the channel can be formed by mixing the “100%” buffer and the “0%” buffer at the ratio of 4 to 1 (v/v), and a region containing denaturant at “20%” in the channel can be formed by mixing the “100%” buffer and the “0%” buffer at the ratio of 1 to 4 (v/v).

The denaturant buffer to be filled in the denaturant buffer reservoir 4 doesn't have to be “100%” denaturant buffer. The denaturant buffer to be filled in the buffer reservoir 5 doesn't have to be “0%” denaturant buffer. For example, in the case that “90%,” denaturant buffer is filled in the denaturant buffer reservoir 4 and “10%” denaturant buffer is filled in the buffer reservoir 5, the concentration-cycle region containing a region contenting denaturant at “90%” and a region containing denaturant at “10%” can be formed in the concentration-cycle channel 2 by injecting two buffers alternatively.

It is also possible to form a cycle comprising three regions, each of which contains different concentration of denaturant. For example, in the case that “100%” denaturant buffer is filled in the denaturant buffer reservoir 4 and “0%” denaturant buffer is filled in the buffer reservoir 5, “100%” and “0%” denaturant regions can be formed by injecting the two buffers into the concentration-cycle channel 12 respectively, and a “10%” denaturant concentration region can be formed by mixing the two buffer at the ratio of 1 to 9 (v/v).

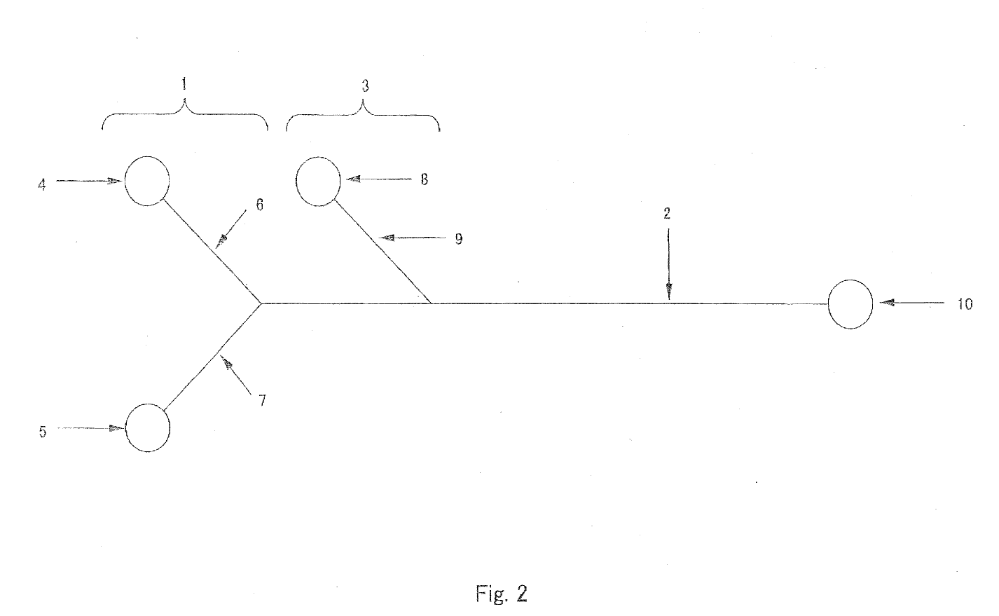

FIG. 2 shows another example of the apparatus of the present invention. This apparatus is suitable in a case where a target nucleic acid moves in the same direction as a denaturant. The sample part comprises the sample reservoir 8 and the sample injection channel 9. The sample reservoir 8 is connected to the concentration-cycle channel 2 through the sample injection channel 9. In this embodiment, the buffer channels 7 and 6 are connected to the concentration-cycle channel at an intersection which is different from the intersection between sample injection channel 9 and the concentration-cycle channel. The product amplified in the concentration-cycle channel 2 is detected at a point of the concentration-cycle channel 2 or collected front the waste reservoir 10. It is also possible to monitor the entire concentration-cycle channel 2.

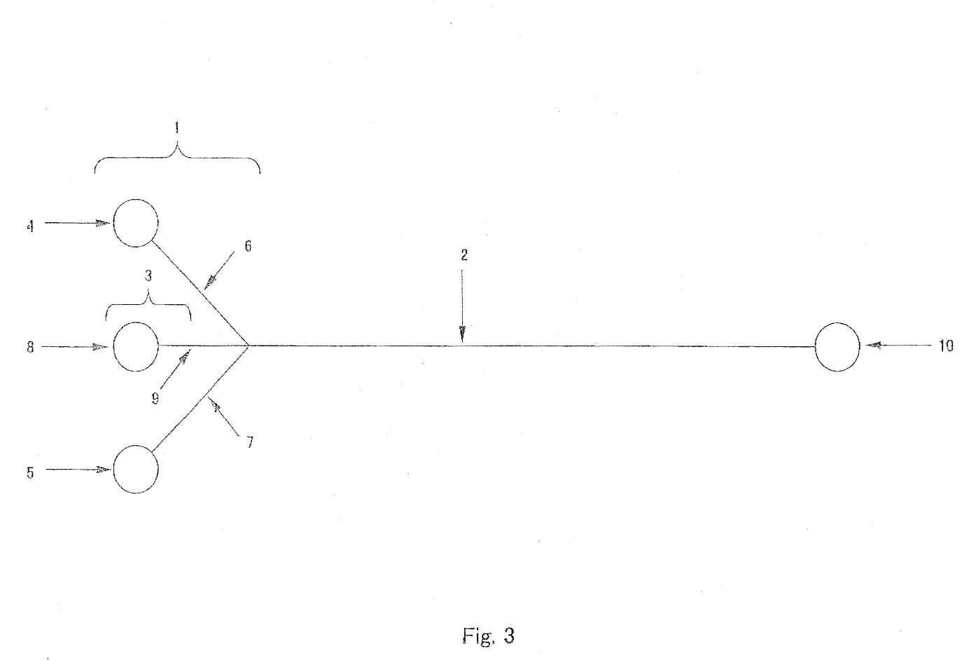

FIG. 3 shows another example of the apparatus of the present invention. This apparatus is also suitable in a case where a target nucleic acid moves in the same direction as a denaturant. The sample part comprises the sample reservoir 8 and the sample injection channel 9. The denaturant buffer channel 6, the buffer channel 7, and the sample injection channel 9 converge on the concentration-cycle channel 2 at one intersection. The product amplified in the concentration-cycle channel 2 is detected at a point of the concentration-cycle channel 2 or collected from the waste reservoir 10. It is also possible to monitor the entire concentration-cycle channel 2

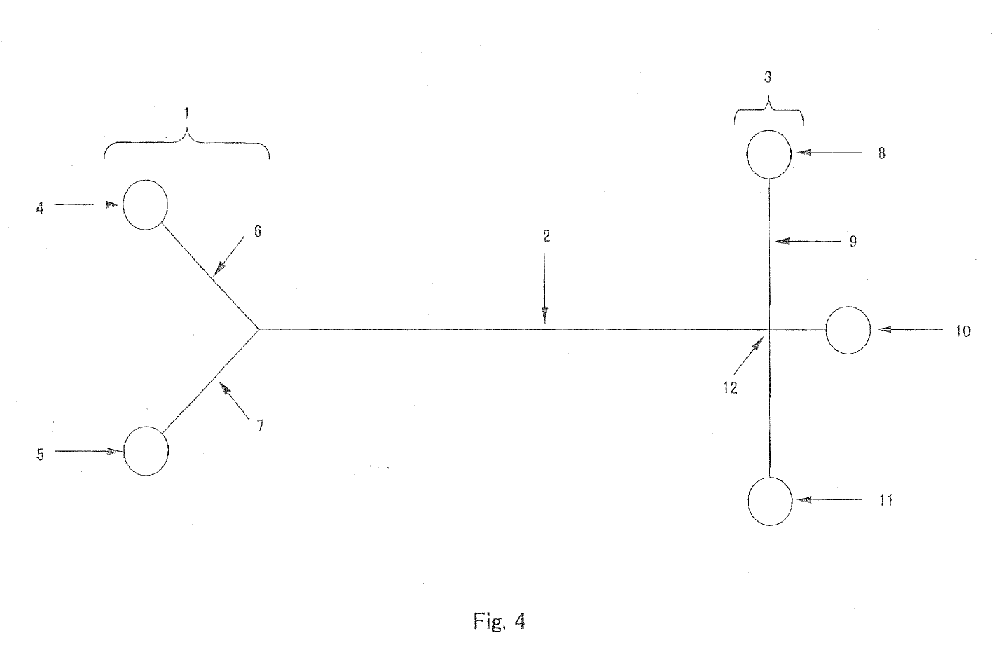

FIG. 4 shows another example of the apparatus of the present invention. This apparatus is suitable in a case where a, target nucleic acid moves in the opposite direction of a denaturant. In addition, this apparatus can precisely inject a certain volume of sample solution by using a sample part disclosed in FIG. 4, which enables precise amount of injections, and therefore is suitable for quantitative amplification. The sample part comprises the sample reservoir 8, the sample injection channel 9, and the sample waste reservoir 11. The sample reservoir 8 is connected to the sample waste reservoir 11 through the sample injection channel 9. The sample injection channel 9 intersects with the concentration-cycle channel 2 at the sample injection region 12. Sample solution containing a target nucleic acid is transported from the sample reservoir 8 to the sample waste reservoir 11 through the sample injection channel 9, and therefore, the target nucleic acid is placed in the sample injection region 12. The plug-shaped target nucleic acid, which is placed in the sample region 12, is injected into the concentration-cycle channel 4. Then, the target nucleic acid goes through the concentration-cycle region, meaning the nucleic acids pass through the concentration-cycle made in the main channel, and in the course of the treatment of the specific nucleic acid sequence is amplified. The amplification product is detected at a point in the channels or collected from the denaturant buffer reservoir 4 and the buffer reservoir 5. It is also possible to monitor all of the channels.

As shown in FIG. 4, there is no injection tool or special procedure for injecting desalting chemicals, which functions as an agent for inactivating an denaturant, into the concentration cycle. Therefore, the step of hybridizing the denatured nucleic acid is done without inactivating the denaturant. Also, the volume of the reaction liquid existing in the concentration-cycle channel 2 can be kept in substantially constant and does not increase in accordance with the progress of the reactions, and therefore, there are no need for adjusting the amount of the injection of reagents in accordance with the amplification.

It is also possible to conduct repetitive amplification. That is, after introducing the hybridized nucleic acid with the extended primer to a concentration-cycle region in the concentration-cycle channel 2 after the first amplification of a nucleic acid so as to pass the hybridized nucleic acid with the extended primer through the concentration cycle region, following steps are needed—(1) to denature the hybridized nucleic acid with the extended primer in the first denaturant concentration region to produce a denatured hybridized nucleic acid with the extended primer, (2) to hybridize the denatured hybridized nucleic acid with the extended primer acid with the primer in the second denaturant concentration region to produce a hybridized nucleic acid, and (3) to extend the primer of the hybridized nucleic acid by the nucleic acid polymerase in the second denaturant concentration region.

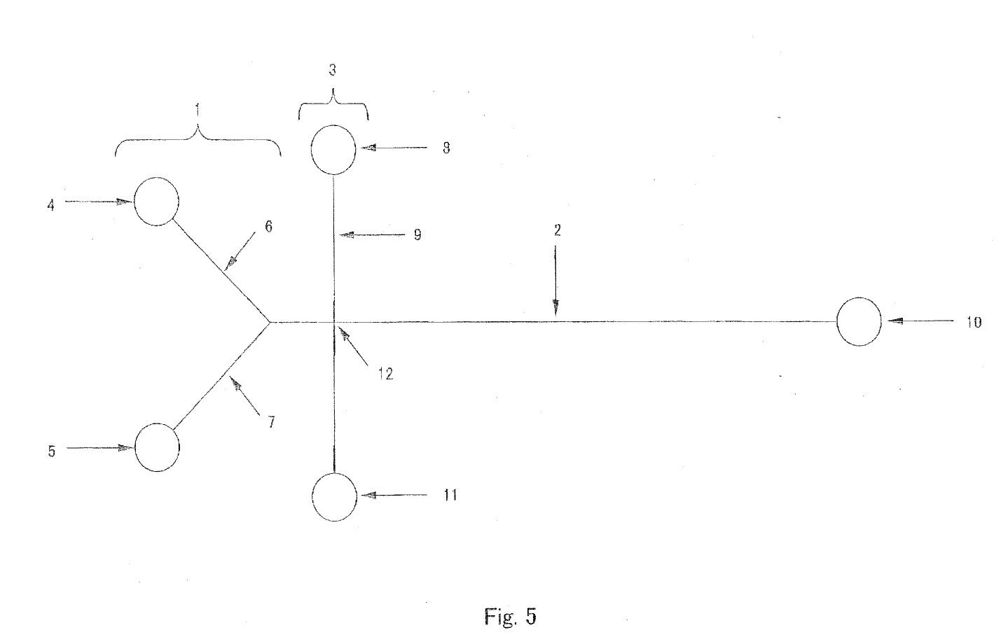

FIG. 5 shows another example of the apparatus of the present invention. This apparatus is suitable in a case where a target nucleic acid moves in the same direction as a denaturant. The amplification product is detected at a point in the concentration-cycle channel 2, or collected from the waste reservoir 10. It is also possible to monitor a concentration of the amplified product across the whole length of the concentration-cycle region channel 2. For example, a fluorescence detector can be used for monitoring a concentration of the amplified product.

The following non-limiting examples explain the invention in more detail.

### EXAMPLE 1

Polymerase chain reaction (PCP) is performed by use of the microchip apparatus as shown in FIG. 4. The microchip is made of poly(methyl methacrylate). The method for making the microchip is as follows. The channels are formed in one substrate by use of hot-embossing. The reservoirs are formed is the other substrate by use of drilling. The two substrate are bonded by using thermal bonding technique. The dimensions of the microchip are 70 mm×35 mm×2 mm. The width and depth of the channels are 100 micron and 25 micron respectively. The diameter of the reservoirs is 3 mm. The reservoirs have Pt electrodes connected with power supplies. The power supplies can be controlled with a personal computer, and therefore the potentials to be applied to the reservoirs are controlled automatically. The primers used in this example are designed to amplify V3 region of 16S ribosomal RNA gene:

(SEQ ID NO. 1)

| | |
|---|---|
| forward primer: | 5′-CCTACGGGAGGCAGCAG-3′; |

(SEQ ID NO. 2)

| | |
|---|---|
| reverse primer: | 5′-ATTACCGCGGCTGCTGG-3′. |

DNA sample solution is prepared as follows. Some colonies of E. coli strain K12 are picked up and resolved in 1 ml TE buffer (10 mM Tris, 1 mM EDTA). The solution is boiled in 100° C. water bath for 10 min, and then centrifuged. The supernatant is diluted 100 times with dilution buffer containing 0.5 μM primers, 0.2 mM of dATP, dTTP, dGTP, and dCTP, 4 mM MgCl₂, 50 mM KCl, SYBR™ Green I and 10 mM Tris-HCl. This diluted solution is used as “DNA sample solution”.

The denaturant buffer reservoir 4 is filled with “100%” denaturant buffer, Taq DNA polymerase, 0.5 μM primers, 0.2 mM of dATP, dTTP, dGTP, and dCTP, 4 mM MgCl2, 50 mM KCl, SYBR™ Green I and 10 mM Tris-HCl. The buffer reservoir, the waste reservoir, and the sample waste reservoir is filled with “0%” denaturant buffer containing Taq DNA polymerase, 0.5 μM primers, 0.2 mM of dATP, dTTP, dGTP, and dCTP, 4 mM MgCl₂, 50 mM KCl, SYBR™ Green I and 10 mM. Tris-HCl. The sample reservoir 8 is filled with the sample solution described above. The sample reservoir 8 is grounded, and a voltage is applied to the sample waste reservoir to transport the sample solution into the sample injection region 12. Then, the waste reservoir 10 is grounded, and voltages are applied to the denaturant buffer reservoir 4 and the buffer reservoir 5 to make concentration-cycle region in the channel 2, and to injection sample solution transported into the sample injection region 12 in the direction of the concentration-cycle region. The voltages applied to the denaturant buffer reservoir 4 and the buffer reservoir 5 are alternatively changed. The amplified product is detected in a low-concentration region with the laser-induced fluorescence detection system comprised of a diode laser and a photomultiplier.

### EXAMPLE 2

Reverse transcription PCR (RT-PCR) is performed by use of the microchip apparatus as shown in FIG. 4. The microchip is made of poly(methyl methacrylate). The method for making the microchip is the same as described in Example 1. RNA sample solution is extracted from methane fermentation sludge. The primers used in this example are the sane as that used in Example 1. LightCycler™ RNA Amplification Kit SYBRGreen™ I is used for this example. The “reaction solution” of the kit contains requisite components for one-step RT-PCR. The denaturant buffer reservoir 4 is filled with the reaction solution containing denaturant at the concentration of “100%”. The buffer reservoir 5, the waste reservoir 10, and the sample waste reservoir 11 are filled with the reaction solution. The sample reservoir is filled with the mixture of the extracted RNA and the reaction solution. The procedures of applying electric, potentials and detecting amplification product are basically the same as Example 1, except that a low-concentration region is formed in the beginning of the concentration-cycle region for reverse transcription reaction.

### EXAMPLE 3

Cycle sequencing reaction is performed by use of the microchip apparatus as shown in FIG. 1. The microchip is made of poly(methyl methacylate). The method for making the microchip is the same as described in the Example 1. The dimensions of the microchip are 70 mm×35 mm×2 mm. The width and depth of the channels are 100 micron and 25 micron respectively. The diameter of the reservoirs is 3 mm. The reservoirs have Pt electrodes connected with power supplies. The power supplies can be controlled with a personal computer, and therefore the potentials to be applied to the reservoirs are controlled automatically.

“DNA sample solution” for PCR is prepared from E. coli as described in the Example 1. The primers used for the PCR are designed to amplify entire 16S ribosomal RNA gene:

(SEQ ID NO. 3)

| | |
|---|---|
| forward primer: | 5′-AGAGTTTGAT CCTGGCTCAG-3′; |

(SEQ ID NO. 4)

| | |
|---|---|
| reverse primer: | 5′-AAAGGAGGTG ATCCAGCC-3′. |

The PCR product is purified with a spin column, and then is diluted to be appropriate concentration for cycle sequencing reaction thereby to obtain “sample solution” for cycle sequencing reaction. The primer used for cycle sequencing reaction is designed to synthesize a part of 16S ribosomal RNA gene:

| | |
|---|---|
| 5′-GTA TTA CCG CGG CTG CTGG-3′. | (SEQ ID NO. 5) |

BigDye™ Terminator Cycle Sequencing Kit is used for this example. The reaction solution of the kit contains requisite components for cycle sequencing reaction. The denaturant reservoir 4 is filled with the reaction solution containing denaturant at the concentration of “100%”. The buffer reservoir 5 is filled with the reaction solution. The sample reservoir 8 is filled with the mixture of the purified PCR product and the reaction solution. The sample reservoir 8 is grounded, and voltages are applied to the denaturant buffer reservoir 4 and the buffer reservoir 5 to make concentration-cycle region in the channel 2, and to inject sample solution into the concentration-cycle region. The voltages applied to the denaturant buffer reservoir 4 and the buffer reservoir 5 are alternatively changed. The product of the cycle sequencing reaction is collected from the denaturant buffer reservoir 4 or the buffer reservoir 5.

1. A method for amplifying a nucleic acid, comprising:
(a) forming in a channel an alternating pattern of a region containing a denaturant in an amount sufficient to denature a nucleic acid, and a region containing a denaturant in an amount sufficient to hybridize a denatured nucleic acid with a primer;
(b) exposing a target nucleic acid to the region containing a denaturant in an amount sufficient to denature a nucleic acid, thereby denaturing the target nucleic acid;
(c) exposing the denatured target nucleic acid to the region containing a denaturant in an amount sufficient to hybridize a denatured nucleic acid with a primer, thereby hybridizing the denatured target nucleic acid with a primer;
(d) allowing the primer hybridized with the target nucleic acid in step (c) to be extended using a nucleic acid polymerase;
(e) exposing the extended product obtained in step (d) to a next region containing a denaturant in an amount sufficient to denature a nucleic acid, thereby denaturing the extended product;
(f) exposing the denatured extended product to a next region containing a denaturant in an amount sufficient to hybridize a denatured nucleic acid with a primer, thereby hybridizing the extended product with a primer; and
(g) allowing the primer hybridized with the extended product in step (f) to be extended using a nucleic acid polymerase.

2. A method according to claim 1, further comprising repeating the steps of (e)-(g) at least one.

3. A method according to claim 1, wherein said region containing a denaturant in an amount sufficient to denature a nucleic acid and said region containing a denaturant in an amount sufficient to hybridize a denatured nucleic acid with a primer are formed by an electrokinetic method.

4. A method according to claims 1, wherein said target nucleic acid and said extended product are exposed to the region containing a denaturant in an amount sufficient to denature a nucleic acid and the region containing a denaturant in an amount sufficient to hybridize a denatured nucleic acid with a primer, by an electrokinetic method.

5. A method according to claim 1, wherein said region containing a denaturant in an amount sufficient to denature a nucleic acid and said region containing a denaturant in an amount sufficient to hybridize a denatured nucleic acid with a primer are formed by a mechanical method.

6. A method according to claim 1, wherein said target nucleic acid and said extended product are exposed to the region containing a denaturant in an amount sufficient to denature a nucleic acid and the region containing a denaturant in an amount sufficient to hybridize a denatured nucleic acid with a primer, by a mechanical method,

7. A method according to claim 1, wherein said nucleic acid polymerase is tolerant to the denaturant.

8. A method according to claim 3, wherein the denaturant and the nucleic acid polymerase are moved at substantially the same velocity by the electrokinetical method.

9. A method for amplifying a nucleic acid sequence contained in a nucleic acid, comprising:
(a) providing a first reservoir, a second reservoir, a main channel, a first channel and a second channel disposed all in a fluidic device, wherein the first channel communicating to the first reservoir and the main channel, and the second channel communicating to the second reservoir and the main channel, and at least one of the reservoirs is filled with a liquid containing a denaturant;
(b) forming in the main channel a concentration-cycle region comprising an alternating pattern of a first region having a denaturant of a first denaturant concentration and a second region having the denaturant of a second denaturant concentration, wherein the first denaturant concentration is higher than the second denaturant concentration and the first and second regions being made by introducing the two liquids alternatively or at different flow ratios;
(c) introducing the nucleic acid to the concentration-cycle region;
(d) passing the nucleic acid through the concentration cycle region;
(e) denaturing the nucleic acid in the first denaturant concentration region to produce a denatured nucleic acid;
(f) hybridizing the denatured nucleic acid with a primer in the second denaturant concentration region to produce a hybridized nucleic acid; and
(g) extending the primer of the hybridized nucleic acid by a nucleic acid polymerase in the second denaturant concentration region.

10. The method according to claim 9, wherein the step of passing the nucleic acid through is performed using electrophoresis by applying a voltage.

11. The method according to claim 9, wherein the step of hybridizing the denatured nucleic acid is done without inactivating the denaturant.

12. The method according to claim 9, further comprising; (h) introducing the hybridized nucleic acid with the extended primer to the concentration cycle region;
(i) passing the hybridized nucleic acid with the extended primer through the concentration cycle region;
(j) denaturing the hybridized nucleic acid with the extended primer in the first denaturant concentration region to produce a denatured hybridized nucleic acid with the extended primer;
(k) hybridizing the denatured hybridized nucleic acid with the extended primer acid with the primer in the second denaturant concentration region to produce a hybridized nucleic acid; and
(l) extending the primer of the hybridized nucleic acid by the nucleic acid polymerase in the second denaturant concentration region.

13. The method according to claim 9, wherein the nucleic acid polymerase is supplied from the first reservoir or the second reservoir.

14. The method according to claim 9, wherein the fluidic device is a microfluidic device.

15. The method according to claim 9, wherein at least one of the first and second channels having a pump to control a flow of the channel to form the concentration-cycle region.

16. The method according to claim 9, wherein the first, second and main channels communicating at a channel intersection.

17. The method according to claim 9, wherein a width of the main channel is in the range of 1 micron to 500 micron.

18. The method according to claim 9, wherein the liquid containing the denaturant is supplied by a pump utilizing electrokinetic effect.

19. The method according to claim 9, wherein the second denaturant concentration is in the range of 0 to 90 percent of the first denaturant concentration.

20. An apparatus for amplifying a nucleic acid, comprising:
(a) a first reservoir, a second reservoir, a first channel and a second channel, wherein the first reservoir and the second reservoir communicate with the first channel and the second channel, respectively, and at least one of the first and the second reservoirs stores a liquid containing a denaturant;
(b) a main channel communicating with the first channel and the second channel, wherein an alternating pattern of a first region containing a first concentration of a denaturant and a second region containing a second concentration of the denaturant, wherein the first concentration is higher than the second concentration and the first and second regions being made by introducing the two liquids stored in the first and the second reservoir alternatively or at different flow ratios. and wherein the nucleic acid is amplified by exposing the nucleic acid to the alternating pattern of the first region and the second region; and
(c) a sample reservoir and a sample channel, wherein the sample reservoir communicates with the sample channel to the main channel, and the sample reservoir stores a nucleic acid to be amplified.

21. An apparatus according to claim 20, further comprising:
a pump introducing the liquid stored in the first and the second reservoirs into the main channel; and
a pump introducing the sample stored in the sample reservoir into the main channel,

22. A method for synthesizing a nucleic acid, comprising:
(a) forming in a channel an alternating pattern of a region containing a denaturant in an amount sufficient to denature a nucleic acid and a region containing a denaturant in an amount sufficient to hybridize a denatured nucleic acid with a primer;
(b) exposing a target nucleic acid to the region containing a denaturant in an amount sufficient to denature a nucleic acid, thereby denaturing the target nucleic acid;
(c) exposing the denatured target nucleic acid to the region containing a denaturant in an amount sufficient to hybridize a denatured nucleic acid with a primer, thereby hybridizing a primer with the denatured target nucleic acid; and
(d) allowing the primer hybridized with the target nucleic acid in step (c) to be extended using a nucleic acid polymerase.

23. A method according to claim 1, further comprising a method for synthesizing said target nucleic acid, which comprises:
(a) forming in a channel an alternating pattern of a region containing a denaturant in an amount sufficient to denature a nucleic acid and a region containing a denaturant in an amount sufficient to hybridize a denatured nucleic acid with a primer:
(b) exposing a pre-target nucleic acid to the region containing a denaturant in an amount sufficient to denature a nucleic acid, thereby denaturing the pre-target nucleic acid;
(c) exposing the denatured pre-target nucleic acid to the region containing a denaturant in an amount sufficient to hybridize a denatured nucleic acid with a primer, thereby hybridizing the denatured pre-target acid with a primer: and
(d) allowing the primer hybridized with the pre-target nucleic acid in step (c) to be extended using a nucleic acid polymerase.
